(19) United States
(12) Plant Patent Application Publication
(10) Pub. No.: US 2004/0006800 P1
(43) Pub. Date: Jan. 8, 2004

(54) Mandarin hybrid tree named "TDE2"

(75) Inventors: Mikeal L. Roose, Riverside, CA (US); Timothy A. Williams, Riverside, CA (US); Robert K. Soost, Inverness, CA (US); James W. Cameron, Salem, OR (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Regents of the University of California, Oakland, CA

(21) Appl. No.: 10/178,000

(22) Filed: Jun. 20, 2002

(51) Int. Cl. A01H 5/00

(52) U.S. Cl. PLT/201

(57) ABSTRACT

A new mandarin hybrid called “TDE2” is distinguished by production of fruit that combines late season maturity, large fruit size, attractive deep orange rind color and virtual absence of seeds with rich fruit flavor.

### BACKGROUND OF THE INVENTION

The pedigree of TDE2 is shown in FIG. 1. In 1973, pollen from Encore mandarin was applied to stigmas of a tetraploid (Temple×4N Dancy) hybrid and the pollinated flowers were bagged to prevent insect pollination. Fruits were collected in winter 1974, seeds extracted from each fruit, and each seed was planted. The chromosome number of each seedling was determined and those identified as triploid seedlings were budded onto Troyer citrange rootstock. The resulting trees were planted in the field in Riverside, Calif. in 1976. These trees were evaluated for tree vigor, bearing, and seediness, fruit flavor, fruit color, and other fruit quality traits from bearing until 1985. Five trees were selected from the original population and repropagated by budding onto C-32 citrange, C-35 citrange, Troyer citrange, and trifoliate orange rootstocks. Two trees of the selection now called TDE2 selection were planting in the field in Riverside in 1987. When they began fruiting (approximately in 1990), these trees were evaluated for the same tree and fruit quality traits as the original trees. In 1987, the selection now called TDE2 was chosen for additional testing because it combined medium or large fruit size, low seed number, rich fruit flavor, deep orange rind and flesh color, and acceptable peelability. Budwood of this selection was tested for viruses and other pathogens by the Citrus Clonal Protection Program and virus-free bud source trees were planted at Lindcove Research and Extension Center, Exeter, Calif. in 1991.

Using this virus-free budwood source, additional trees were propagated and planted at several California locations between 1993 and 1996. These included two locations in the Coachella Valley (Thermal, 73 trees, and the Coachella Valley Agricultural Research Station-CVARS, 4 trees), Ojai (12 trees) and Santa Paula (6 trees) in Ventura Co., and Valley Center (11 trees) in San Diego Co. These trial plantings provide most of the available data on TDE2. Several different rootstocks have been used in these evaluations, mostly Carrizo citrange, C35 citrange, and Schaub rough lemon. The trees in Valley Center are topworked Valencia orange on Troyer citrange rootstock. In general, no major effects of these rootstocks on fruit quality of TDE2 were observed, and no incompatibilities have been evident, but longevity of trees on various rootstocks is not known.

### BRIEF SUMMARY OF THE INVENTION

The present invention provides a novel mandarin hybrid having the characteristics described and illustrated herein. The hybrid, TDE2, produces fruit that combines late season maturity, large fruit size, attractive deep orange rind color and virtual absence of seeds with rich fruit flavor.

### BRIEF DESCRIPTION OF THE DRAWINGS

FIG. 1 illustrates the pedigree of TDE2. All cultivars are C. reticulata except orange, which is C. sinensis.

FIG. 2 illustrates, clockwise from top left: a nine-year-old tree of TDE2 on Carrizo rootstock; fruit on tree; branching pattern; flower buds; flowers; leaves; and shoots.

FIG. 3 illustrates fruit of TDE2 sampled from nine-year-old tree on Carrizo rootstock.

FIGS. 4A-E illustrate the solids:acid ratio of TDE2 at five California locations over five years. Points plotted are means of all samples collected on a given date. Solid lines connect means for sampling dates within the same season. The dashed line is a liner regression of solids:acid on sampling date using data from all years. The regression equation and r² value are shown in each figure.

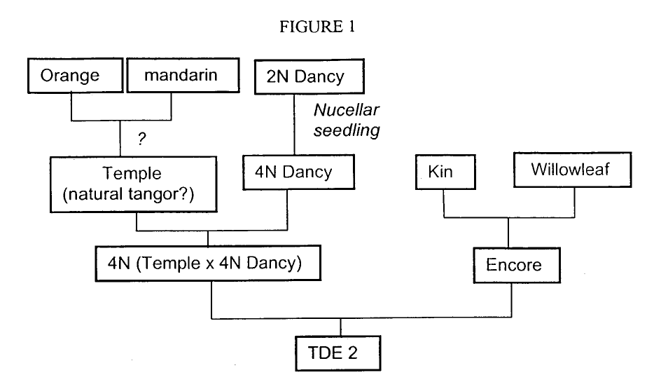

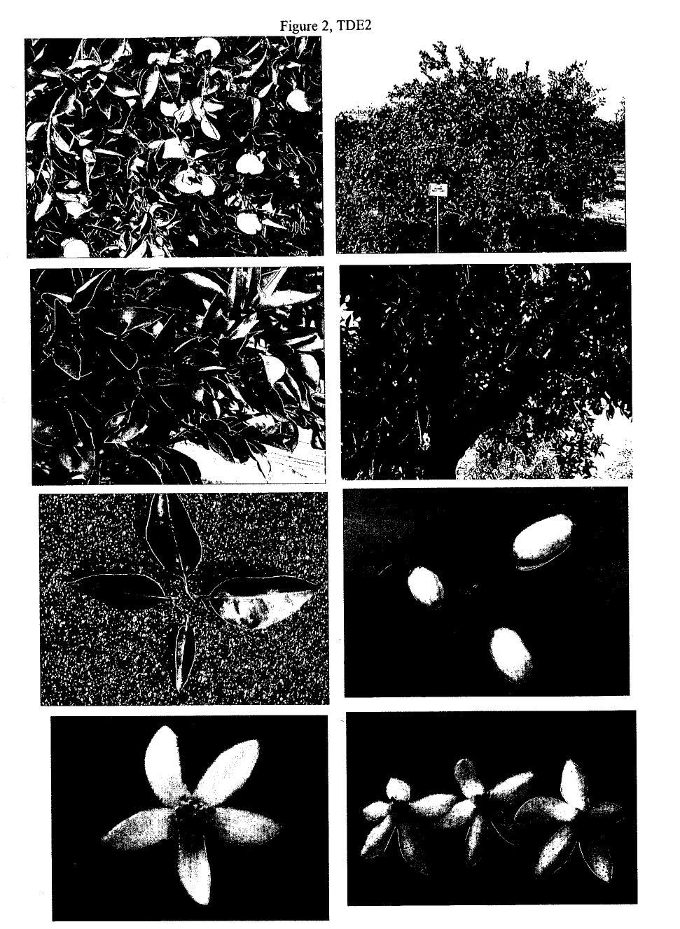

### DETAILED DESCRIPTION OF THE INVENTION

Tree shape is approximately sphereoid (FIG. 2), rather similar to that of orange trees. The trees have not been noted as particularly susceptible to any diseases and, based on a freeze in 1999, appeared only slightly more cold hardy than oranges of similar age. Leaves (FIG. 2) are simple, brevipetiolate, lanceolate, with entire or slightly sinuate margins. The petiole shape is narrow and linear in shape. In comparison with most old-line citrus cultivars, trees of TDE2 are very thorny, with normal branches having medium length (15 mm) thorns at about 50% of the nodes, and watersprouts having long (31 mm) thorns at about 73% of nodes. Thorniness will probably decrease as the cultivar ages.

Flowers of TDE2 are typically hermaphroditic, with white petals and yellow anthers (FIG. 2). Trees flower from early April into May at most locations. Pollen is somewhat sparse, with 10% viability as estimated in an in vitro germination test. Pollen tube growth was also less vigorous than that of fertile, diploid mandarins.

If sufficient fruit was available, 10-fruit samples were collected from each location two or three times each year beginning in 1997 or 1998. Generally samples were collected from two or three trees on each sampling date. These fruit were evaluated in Riverside for a range of traits as summarized in Table 1.

TABLE 1
Fruit characteristics of TDE2 averaged over 5 locations and 4
seasons. Samples were collected from mid-January to early May
at Santa Paula, Ojai, and Valley Center, and from mid-November
to early April at Thermal and CVARS. “N” indicates the
total number of fruit samples analyzed. Data shown are averaged
over fruit from trees on various rootstocks.

| Trait | N | Min | Max | Mean | SD |
|---|---|---|---|---|---|
| Fruit height (mm) | 149 | 38.7 | 80.0 | 58.4 | 6.46 |
| Fruit width (mm) | 149 | 57.5 | 91.6 | 74.5 | 5.71 |
| Fruit height:width | 149 | 0.52 | 0.94 | 0.78 | 0.064 |
| Rind color rating^{d} | 149 | 4.5 | 13.0 | 11.8 | 1.08 |
| Rind texture^{b} | 149 | 2.2 | 7.5 | 3.7 | 0.68 |
| Neck rating^{c} | 149 | 0 | 2.50 | 0.47 | 0.66 |
| Peelability rating^{d} | 149 | 6.00 | 9.50 | 7.91 | 0.736 |
| Rind thickness (mm) | 149 | 3.00 | 6.00 | 3.86 | 0.626 |
| Seeds per fruit | 149 | 0 | 0.40 | 0.02 | 0.065 |
| Fruit weight (g) | 149 | 103.5 | 370.0 | 184.5 | 45.12 |
| Juice content (%) | 149 | 32.4 | 62.5 | 49.4 | 4.911 |
| Soluble solids (%) | 146 | 7.50 | 15.55 | 12.38 | 1.749 |
| Acid (%) | 145 | 0.62 | 2.64 | 1.22 | 0.341 |
| Solids:acid | 145 | 4.10 | 22.90 | 10.84 | 3.204 |

^{a}Visual rating on a scale of 0-13; 0 = green, 13 = red-orange
^{b}Visual rating on a scale of 1-8; 1 = very smooth, 8 = extremely coarse
^{c}Visual rating on a scale of 0-3; 0 = no trace of neck, 3 = neck with a diameter at least 50% of fruit diameter
^{d}Subjective rating of ease of peeling a single fruit; 1 = very difficult, 10 = a fruit with completely separated rind and segments. Fruit with ratings of 7 or higher would be relatively easy to peel.

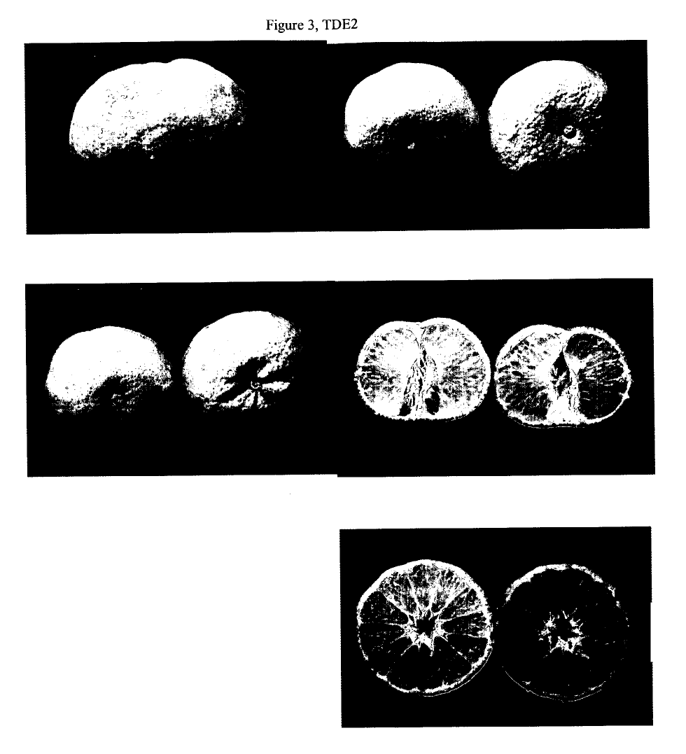

Based on this data, TDE2 fruit are oblate in shape, with little or no neck (FIG. 3). The average fruit size is large for a mandarin (classed as Mammoth by California state standards). Rind color is deep orange with color scores of L=65.4, C=65.5, H=68.5 for fruit harvested in Riverside on Feb. 10. The rind texture is variable, depending on tree age and crop. For older trees with a moderate to heavy crop, rind texture is slightly pitted, with depressed oil glands. The rind of fruit from trees with very light crops is often excessively rough or bumpy. The fruit base (stalk end) is slightly concave (FIG. 3), and the apex is truncate with a slight depression in the stylar end and a small (4 mm), occasionally open stylar scar. The rind is fairly easy to peel when fruit are mature, but can be more adherent early in the season. Flesh color is deep orange.

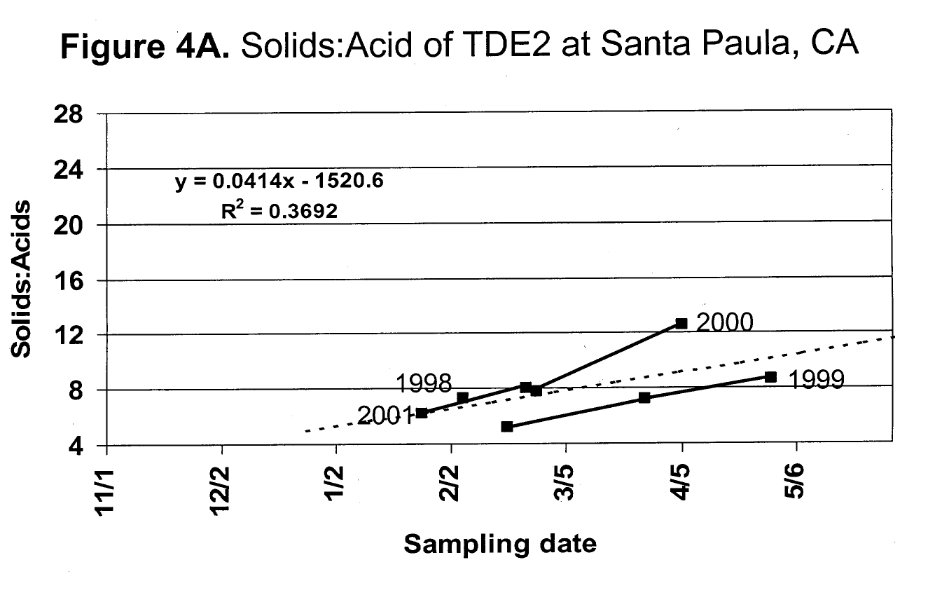

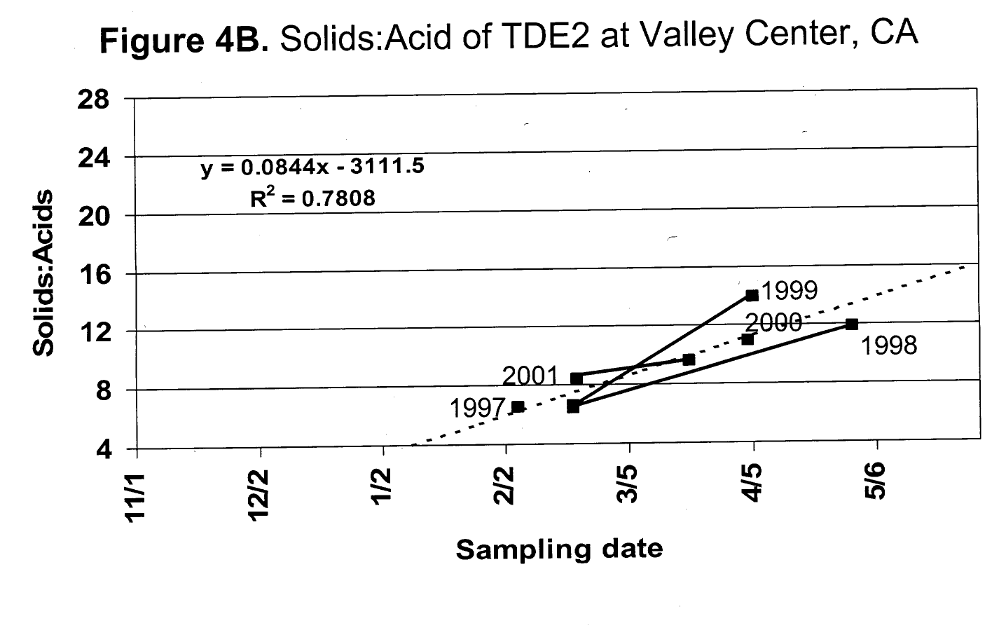

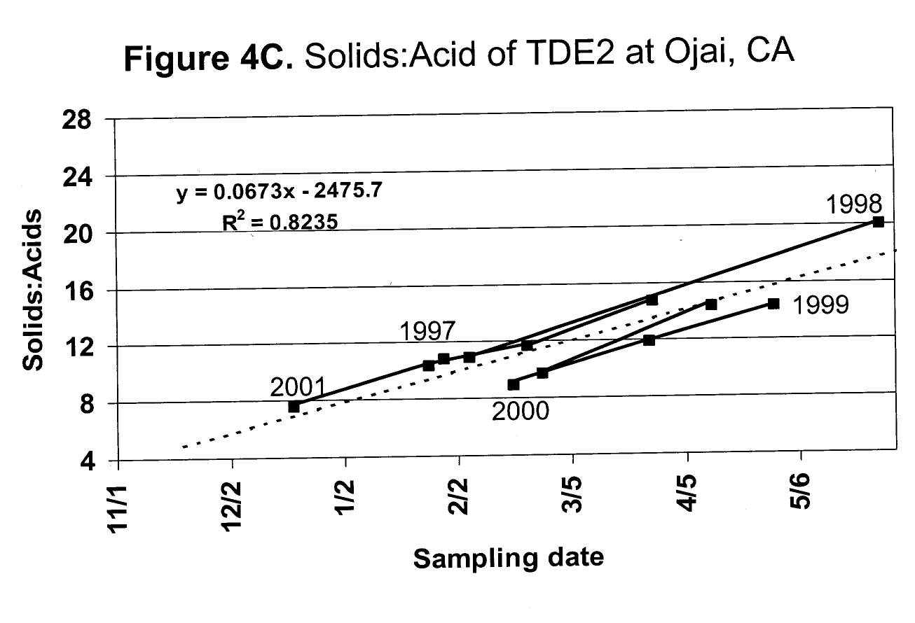

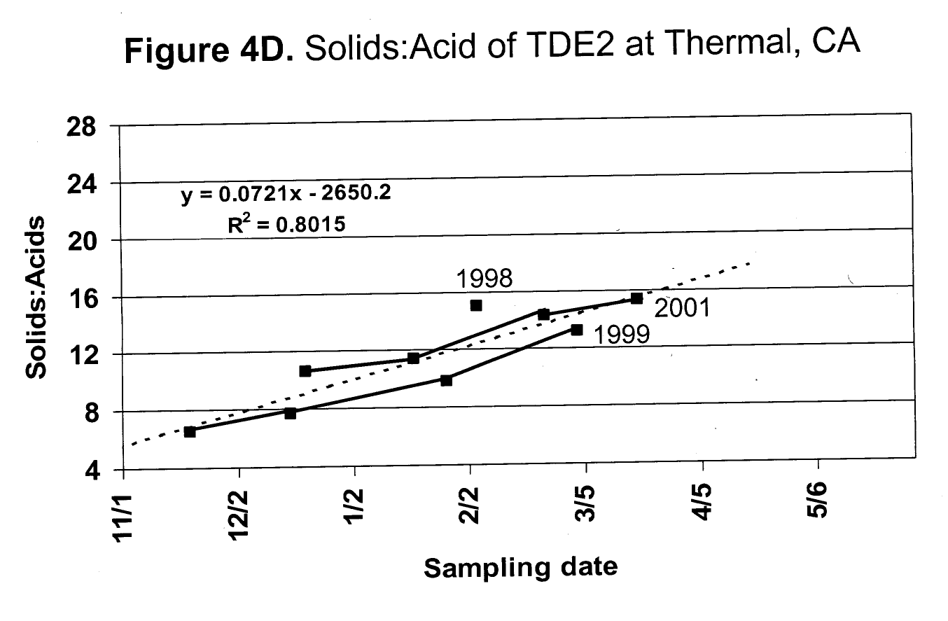

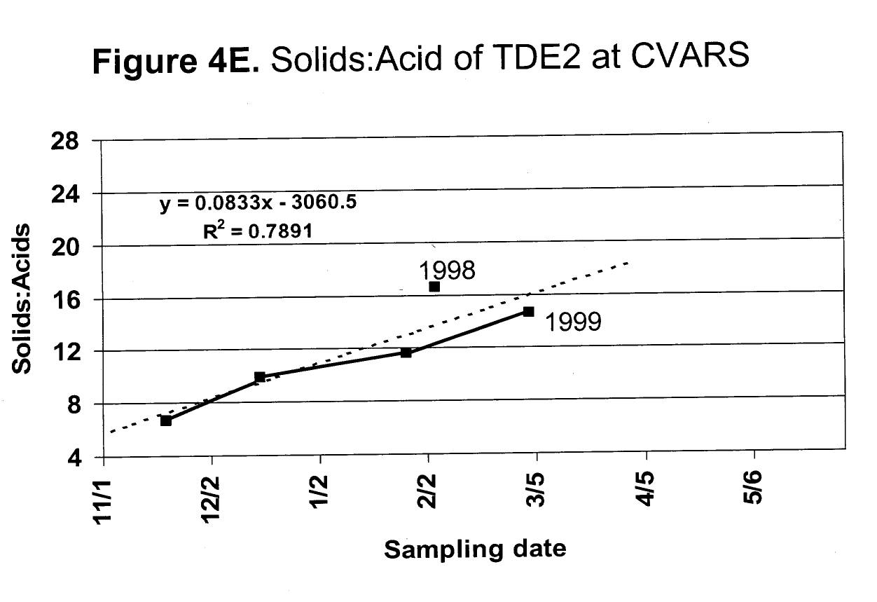

Important determinants of maturity date for citrus fruit are the solids:acid ratio and juice content. Using data for all years, juice content did not show a statistically significant correlation with sampling date at any of the 5 locations. This indicates that there was not generally any significant drying of fruit during the sampling period. Solids:acids ratio was significantly correlated with sampling date at all location except Santa Paula (FIG. 4). Using these regressions, the estimated dates on which fruit reached an 8:1 solids:acid ratio was December 6 for Thermal, January 2 for Ojai, February 20 for Valley Center, and March 5 for Santa Paula. The limited data for CVARS are consistent with those for the climatically similar Thermal site.

Yield of TDE2 was evaluated from visual ratings of crop relative to tree size at each location from 1998-99 to 2001-2002. The rating scale ranged from 0 (no crop) to 5 (very heavy crop). The topworked trees in Valley Center showed the highest and most consistent crops, ranging between 3 and 4 over the 4 years studied. Crops at Ojai were also good, being 2.5 or greater in all years. At Santa Paula, crop ratings indicated alternate bearing, with average values of 2.17, 3.67, 1.17, and 3.50 from 1998-99 to 2001-2002, respectively. Trees planted at Thermal in 1994 showed similar behavior, but with lower values of 1.83, 0.50, 2.40, and 1.40, while those planted in 1996 had crops of 0, 0, 2.87, and 1.5.

Because TDE2 is a late maturing fruit, it is likely that trees will show a fairly strong tendency to alternate bearing, and this is supported by the data for some locations.

During the 1998-99 season, fruit of TDE2 and Gold Nugget, another late season mandarin with few seeds, were harvested on April 12 from Valley Center and evaluated by a taste panel before and after storage at two different temperatures. Fruit were rated on a 9 point scale, where a score of 1 is “Dislike extremely”, 5 is “Neither dislike or like”, and 9 is “Like extremely”. Results (Table 2) show that before storage TDE2 was preferred to Gold Nugget based on visual appearance, peelability, and taste, with good overall scores for these traits. After storage at 20.5 C. for 11 days, both cultivars improved in visual appeal and peelability, but only Gold Nugget improved in taste. Storage for 12 days at 3.4 or 5.6 C. followed by 7 days at 13.3 C. did not greatly affect any of the ratings, but taste of both cultivars was decreased slightly in cold storage at 3.4 C. Waxed fruit were similar to unwaxed for nearly all traits. Storage at 5.6 C. decreased visual appeal of TDE2 slightly while storage at 20.5 C. increased visual appeal, peelability, and taste scores. Overall, these data indicate that TDE2 fruit can be stored without greatly affecting visual appeal or taste.

TABLE 2
Sensory evaluation of ‘Gold Nugget’ and ‘TDE 2’
harvested Apr. 12, 1999 from Valley Center, CA.

| | Visual Evaluation | | | |
|---|---|---|---|---|
| | Gold | Gold | | |
| | Nugget | Nugget | TDE 2 | TDE 2 |
| | −wax | +wax | −wax | +wax |
| Initial | | | | |
| Mean | 4.3 | 5.0 | 6.8 | 7.0 |
| SD | 2.1 | 2.0 | 1.6 | 1.5 |
| 11 days @ 68 F. | | | | |
| Mean | 5.4 | 6.2 | 7.3 | 7.9 |
| SD | 1.6 | 1.5 | 1.4 | 0.9 |
| 12 days @ 37 F. + 7 days @ 55 F. | | | | |
| Mean | 5.3 | 5.7 | 6.8 | 7.2 |
| SD. | 2.5 | 2.1 | 1.3 | 1.5 |
| 12 days @ 41 F. + 7 days @ 55 F. | | | | |
| Mean | 5.5 | 5.7 | 7.4 | 7.1 |
| SD | 2.3 | 2.1 | 1.4 | 1.4 |
| | Peelability Evaluation | | | |
| | Gold | Gold | | |
| | Nugget | Nugget | TDE 2 | TDE 2 |
| | −wax | +wax | −wax | +wax |
| Initial | | | | |
| Mean | 4.6 | 4.0 | 7.0 | 6.8 |
| SD | 1.7 | 1.9 | 1.3 | 1.5 |
| 11 days @ 68 F. | | | | |
| Mean | 5.3 | 5.4 | 7.6 | 7.5 |
| SD | 2.1 | 2.2 | 1.1 | 0.8 |
| 12 days @ 37 F. + 7 days @ 55 F. | | | | |
| Mean | 5.2 | 5.6 | 7.1 | 7.2 |
| SD. | 1.7 | 1.9 | 1.6 | 1.5 |
| 12 days @ 41 F. + 7 days @ 55 F. | | | | |
| Mean | 6.1 | 5.2 | 7.4 | 7.3 |
| SD | 1.4 | 1.7 | 1.4 | 1.5 |

| Taste Evaluation | | | |
|---|---|---|---|
| Gold | Gold | | |
| Nugget | Nugget | TDE 2 | TDE 2 |
| −wax | +wax | −wax | +wax |

Initial

| Mean | 5.4 | 5.3 | 6.5 | 6.8 |
|---|---|---|---|---|
| SD | 2.0 | 2.6 | 1.6 | 1.7 |
| 11 days @ 68 F. | | | | |
| Mean | 7.3 | 6.8 | 6.5 | 5.7 |
| SD | 1.7 | 1.9 | 1.7 | 1.6 |
| 12 days @ 37 F. + 7 days @ 55 F. | | | | |
| Mean | 6.1 | 6.0 | 5.8 | 6.3 |
| SD. | 1.9 | 2.4 | 1.6 | 1.5 |
| 12 days @ 41 F. + 7 days @ 55 F. | | | | |
| Mean | 6.5 | 6.6 | 6.9 | 6.5 |
| SD | 1.6 | 1.6 | 1.4 | 1.7 |

Two siblings of TDE2, “TDE3” and “TDE4,” were compared to TDE2. TDE2 is distinct from these cultivars in having the latest maturity date, the largest fruit size, a more oblate shape than TDE3, and distinctive flavor. The rind color of TDE2 is usually paler orange than that of TDE4. Trees or fruit of TDE2 can be distinguished from those of other mandarins, including TDE3 and TDE4, using simple sequence repeat (SSR) DNA markers. Using TDE2 DNA as template, PCR primer set TAA15 (F=GAAAGGGTTACTTGACCAGGC, R=CTTCCCAGCTGCACAAGC) amplified a band of 185 bp, while TDE3 and TDE4 both had two bands of 185 and 200 bp. Bands amplified with TAA15 combined with those amplified with either CAC15 (F=TAAATCTCCACTCTGCAAAAGC, R=GATAGGAAGCGTCGTAGACCC) or TAA33 (F=GGTACTGATAGTACTGCGGCG, R=GCTAATCGCTACGTCTTCGC) distinguished TDE2 from the following cultivars: Dancy, Temple, Encore, King, Willowleaf, Wilking, Gold Nugget, Pixie, W. Murcott, Ellendale, Hernandina Clementine, Fortune, Kara, Kinnow, Murcott, Nova, and Ponkan.

Vigor of TDE2 trees has varied greatly across locations. In the two desert locations, canopy volumes of 7-year-old trees averaged 41.1 and 28.8 m³, and 5 year-old trees averaged were 9.7 m³. In contrast, at the cooler Santa Paula and Ojai locations, 7-year-old trees averaged 6.3 and 6.1 m³. Trees in the desert locations have averaged somewhat less crop relative to tree size, perhaps contributing to greater vegetative growth. Size of the topworked trees in Valley Center has not been measured since they are not comparable to trees in other locations, but in general the topworked trees are quite vigorous. Rootstocks had some effect on trees size. At Thermal, trees on Volkamer lemon had canopy volumes about twice that of trees on Carrizo or C35 citranges. Trees on Schaub rough lemon were usually larger than those on Carrizo or C35 citranges. No evidence of stock-scion incompatibilities was evident, but trees are still relatively young.

TDE2 can be propagated on many available citrus rootstocks by budding. Because of the high level of thorniness, great care should be taken to select budwood from upper-canopy branches having no thorns. Tree spacing in field plantings will depend on vigor of the rootstock. Trees can be grown with pollinizer cultivars such as Minneola, Valencia orange, unrelated mandarins (not Temple, Dancy, Encore or other TDE hybrids) that produce viable pollen. Maturity dates will vary with location, probably depending on the number of heat units and soil conditions.

As in some other mandarins, sprays with gibberellic acid may increase fruit set when pollinizers and/or pollinators are inadequate.

What is claimed is:

1. A new and distinct variety of mandarin hybrid tree having the characteristics described and illustrated herein.


SEQUENCE LISTING (6 sequences)

SEQ ID NO: 1 - DNA, 21 bp, Artificial Sequence
  Description of Artificial SequencePCR amplification primer TAA15 F
gaaagggttacttgaccaggc

SEQ ID NO: 2 - DNA, 18 bp, Artificial Sequence
  Description of Artificial SequencePCR amplification primer TAA15 R
cttcccagctgcacaagc

SEQ ID NO: 3 - DNA, 22 bp, Artificial Sequence
  Description of Artificial SequencePCR amplification primer CAC15 F
taaatctccactctgcaaaagc

SEQ ID NO: 4 - DNA, 21 bp, Artificial Sequence
  Description of Artificial SequencePCR amplification primer CAC15 R
gataggaagcgtcgtagaccc

SEQ ID NO: 5 - DNA, 21 bp, Artificial Sequence
  Description of Artificial SequencePCR amplification primer TAA33 F
ggtactgatagtactgcggcg

SEQ ID NO: 6 - DNA, 20 bp, Artificial Sequence
  Description of Artificial SequencePCR amplification primer TAA33 R
gctaatcgctacgtcttcgc